(19) United States
(12) Patent Application Publication
(10) Pub. No.: US 2023/0233852 A1
(43) Pub. Date: Jul. 27, 2023

(54) WALKING ASSIST DEVICE

(71) Applicants: OSAKA UNIVERSITY, Suita-shi (JP); JTEKT CORPORATION, Kariya-shi (JP)

(72) Inventors: Matija MILOSEVIC, Suita-shi (JP); Koshi SHIBAGAKI, Suita-shi (JP); Taishin NOMURA, Suita-shi (JP); Yoshitaka KAWAKAMI, Obu-shi (JP); Yoshitaka YOSHIMI, Kariya-shi (JP)

(73) Assignees: OSAKA UNIVERSITY, Suita-shi (JP); JTEKT CORPORATION, Kariya-shi (JP)

(21) Appl. No.: 18/148,089

(22) Filed: Dec. 29, 2022

(30) Foreign Application Priority Data
Jan. 21, 2022 (JP) 2022-007724

(51) Int. Cl. A61N 1/36; A61N 1/04

(52) U.S. Cl. CPC A61N 1/36003; A61N 1/0476; A61N 1/36031; A61B 5/1123

(57) ABSTRACT

A walking assist device includes a plurality of electrodes disposed on a surface of a leg of a user, a stimulation applier configured to apply, to the electrodes, a voltage for applying electrical stimulation to the muscles, and a control unit configured to control the stimulation applier to apply the voltage to one or more target electrodes selected from among the electrodes. The control unit controls the stimulation applier such that a combination of electrodes selected as the one or more target electrodes is different for each of a plurality of motion phases included in a motion cycle of the leg during the walking motion.

### CROSS-REFERENCE TO RELATED APPLICATION

This application claims priority to Japanese Patent Application No. 2022-007724 filed on Jan. 21, 2022, incorporated herein by reference in its entirety.

### BACKGROUND

### 1. Technical Field

The disclosure relates to a walking assist device.

### 2. Description of Related Art

U.S. Pat. No. 5,643,332 discloses a device that assists a walking motion by functional electrical stimulation (FES). The device (FES device) includes electrodes for stimulating the tibialis anterior muscle of a user, a tilt sensor for detecting the angle of the lower leg, and a control circuit for controlling the electrodes and the tilt sensor. The control circuit controls the electrodes to stimulate the tibialis anterior muscle in response to the output of the tilt sensor. Thereby, the FES device assists the walking motion of the user.

### SUMMARY

The FES device is often used to assist the motion of patients with severe disabilities, such as stroke and spinal cord injury. Incidentally, the FES has a function to work muscles by electrical stimulation, and for users other than patients with severe disabilities, the FES is conceivable to be used for walking training and walking assist to suppress deterioration of walking ability, for example.

However, when the FES device is used for walking training or walking assist, the muscle to be stimulated is merely the tibialis anterior muscle, and as a consequence, there is a problem that it is difficult to perform appropriate walking assist.

An aspect of the disclosure relates to a walking assist device. The walking assist device includes a plurality of electrodes disposed on a surface of a leg of a user and corresponding to a plurality of muscles used for a walking motion, a stimulation applier configured to apply, to the electrodes, a voltage for applying electrical stimulation to the muscles, and a control unit configured to control the stimulation applier to apply the voltage to one or more target electrodes selected from among the electrodes. The control unit is configured to control the stimulation applier such that a combination of electrodes selected as the one or more target electrodes is different for each of a plurality of motion phases included in a motion cycle of the leg during the walking motion.

According to the aspect of the disclosure, walking assist can be performed appropriately.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a diagram showing a walking assist device according to an embodiment worn on legs of a user;

FIG. 2 is a side view of a leg of the user wearing the walking assist device;

FIG. 3 is a block diagram showing a configuration of a controller;

FIG. 4 is a diagram showing motion phases included in a motion cycle of the leg;

FIG. 5 is a diagram showing a state of the leg in four motion phases;

FIG. 6 is a graph showing an example of temporal changes in an angular velocity of a right thigh obtained by a processing unit;

FIG. 7 is a flowchart showing an example of a determination process;

FIG. 8 is a flowchart showing an example of a change process;

FIG. 9 is a time chart showing a state of each of electrodes controlled by the change process;

FIG. 10 is a diagram showing muscles to which electrical stimulation is applied when electrodes are controlled according to the time chart of FIG. 9, showing muscles to which electrical stimulation is applied for each motion phase;

FIG. 11 is a flowchart showing an example of a change process according to another embodiment;

FIG. 12 is a time chart showing a state of each of the electrodes controlled by the change process according to the other embodiment; and

FIG. 13 is a diagram showing muscles to which electrical stimulation is applied when electrodes are controlled according to the time chart of FIG. 12.

### DETAILED DESCRIPTION OF EMBODIMENTS

### Overview of Embodiment

First, the contents of the embodiments will be listed and described.

A walking assist device according to an embodiment includes a plurality of electrodes disposed on a surface of a leg of a user and corresponding to a plurality of muscles used for a walking motion, a stimulation applier that applies, to the electrodes, a voltage for applying electrical stimulation to the muscles, and a control unit that controls the stimulation applier to apply the voltage to one or more target electrodes selected from among the electrodes. The control unit controls the stimulation applier such that a combination of electrodes selected as the one or more target electrodes is different for each of a plurality of motion phases included in a motion cycle of the leg during the walking motion.

With the configuration, the combination of electrodes selected as the target electrodes to which the voltage is applied is different for each of the motion phases, and thus electrical stimulation can be selectively applied to a plurality of muscles of the leg to be moved during the walking motion. As a result, walking assist can be performed appropriately.

The above-described walking assist device further includes a detector that detects a motion state of the leg, and desirably, the control unit performs a determination process for determining, based on an output of the detector, which of the motion phases is the current motion phase of the leg during the walking motion. In this case, the processing unit can determine the current motion phase of the leg based on the motion state of the leg.

In the walking assist device, desirably, the motion state of the leg includes an angular velocity of the thigh of the leg. In this case, the current motion phase of the leg can be determined by detecting at least the angular velocity of the thigh.

In the walking assist device, desirably, the muscles include a biceps femoris muscle, a vastus medialis muscle, a soleus muscle, and a tibialis anterior muscle. In this case, electrical stimulation can be applied to appropriate muscles during the walking motion.

In the walking assist device, desirably, the motion phases include the following four motion phases. A transition phase is a motion phase in which the leg transitions from a swing state to a stance state, a kick phase is a motion phase in which the leg in the stance state kicks backward, a pre-swing phase is a motion phase in which, immediately before the leg transitions from the stance state to the swing state, right and left legs are in contact with a ground, and a toe raise phase is a motion phase in a state in which a toe of the leg in the swing state is raised.

In this case, electrical stimulation can be applied at appropriate timing during the walking motion.

In the walking assist device, the muscles include a biceps femoris muscle, a vastus medialis muscle, a soleus muscle, and a tibialis anterior muscle, and the motion phases include the following four motion phases. In the transition phase to be described below, electrodes disposed corresponding to the biceps femoris muscle, the vastus medialis muscle, and the tibialis anterior muscle among the electrodes are the target electrodes. In the kick phase to be described below, an electrode disposed corresponding to the soleus muscle among the electrodes is the target electrode. In the pre-swing phase to be described below, none of the electrodes is the target electrode. In the toe raise phase to be described below, an electrode disposed corresponding to the tibialis anterior muscle among the electrodes is the target electrode. The transition phase is a motion phase in which the leg transitions from a swing state to a stance state, the kick phase is a motion phase in which the leg in the stance state kicks backward, the pre-swing phase is a motion phase in which, immediately before the leg transitions from the stance state to the swing state, right and left legs are in contact with a ground, and the toe raise phase is a motion phase in a state in which a toe of the leg in the swing state is raised.

With the configuration, electrical stimulation can be applied to muscles to be moved during the walking motion at appropriate timing.

Further, in the walking assist device, the muscles include a biceps femoris muscle, a vastus medialis muscle, a soleus muscle, and a tibialis anterior muscle, and the motion phases include the following four motion phases. In the transition phase to be described below, electrodes disposed corresponding to the biceps femoris muscle, the vastus medialis muscle, the soleus muscle, and the tibialis anterior muscle among the electrodes are the target electrodes. In the kick phase to be described below, an electrode disposed corresponding to the soleus muscle among the electrodes is the target electrode. In the pre-swing phase to be described below, an electrode disposed corresponding to the tibialis anterior muscle is the target electrode. In the toe raise phase to be described below, an electrode disposed corresponding to the tibialis anterior muscle among the electrodes is the target electrode. The voltage applied to the electrode disposed corresponding to the soleus muscle in the transition phase is lower than the voltage applied to the electrode disposed corresponding to the soleus muscle in the kick phase. The voltage applied to the electrode disposed corresponding to the tibialis anterior muscle in the pre-swing phase is lower than the voltage applied to the electrode disposed corresponding to the tibialis anterior muscle in the toe raise phase. The transition phase is a motion phase in which the leg transitions from a swing state to a stance state, the kick phase is a motion phase in which the leg in the stance state kicks backward, the pre-swing phase is a motion phase in which, immediately before the leg transitions from the stance state to the swing state, right and left legs are in contact with a ground, and the toe raise phase is a motion phase in a state in which a toe of the leg in the swing state is raised.

Even in the configuration, electrical stimulation can be applied to muscles to be moved during the walking motion at appropriate timing.

Details of Embodiment

Hereinafter, a preferred embodiment will be described with reference to the drawings.

Regarding Overall Structure

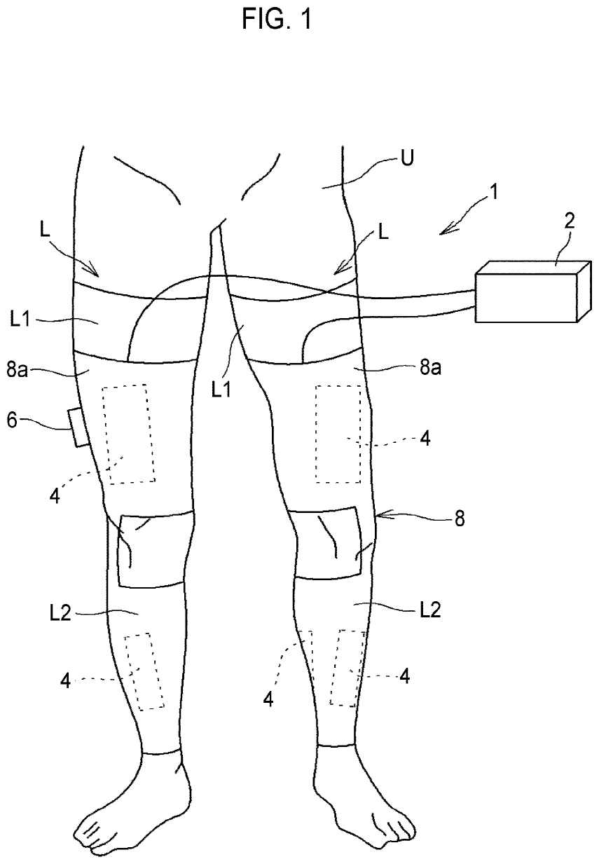

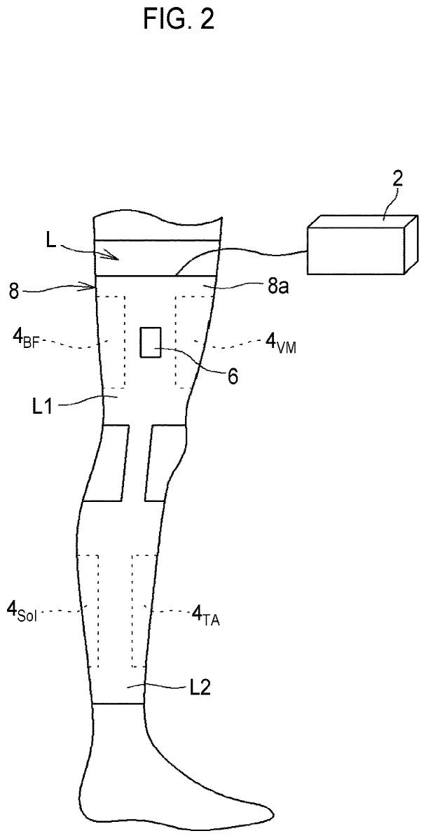

FIG. 1 is a diagram showing a walking assist device according to an embodiment worn on the legs of a user, and FIG. 2 is a side view of a leg of the user wearing the walking assist device. The walking assist device 1 is worn on a pair of right and left legs L of a user U, and has a function of performing walking training and walking assist for the user U by functional electrical stimulation (FES).

The walking assist device 1 includes a controller 2, a plurality of electrodes 4, a detector 6, and a wearing tool 8. The electrodes 4 are attached and disposed on the surfaces of the legs L. The electrodes 4 are electrodes for applying electrical stimulation to muscles from surface of the legs L. The electrodes 4 are connected to the controller 2. The electrodes 4 apply electrical stimulation to the muscles of the legs L with a voltage applied from the controller 2. On each of the legs L, four electrodes 4 are disposed corresponding to the muscles used for the walking motion. In the present embodiment, the biceps femoris muscle, vastus medialis muscle, soleus muscle, and tibialis anterior muscle are targeted as the muscles used for the walking motion.

As shown in FIG. 2, the four electrodes 4 disposed on each of the legs L include a first electrode 4_{BF}, a second electrode 4_{VM}, a third electrode 4_{So1}, and a fourth electrode 4_{TA}. The first electrode 4_{BF} is disposed at the rear portion of a thigh L1. The first electrode 4_{BF} is disposed corresponding to the biceps femoris muscle of the thigh L1, and applies electrical stimulation to the biceps femoris muscle. The second electrode 4_{VM} is disposed at the front portion of the thigh L1. The second electrode 4_{VM} is disposed corresponding to the vastus medialis muscle of the thigh L1, and applies electrical stimulation to the vastus medialis muscle. The third electrode 4_{So1} is disposed at the rear portion of a lower leg L2. The third electrode 4_{So1} is disposed corresponding to the soleus muscle of the lower leg L2, and applies electrical stimulation to the soleus muscle. The fourth electrode 4_{TA} is disposed at the front portion of the lower leg L2. The fourth electrode 4_{TA} is disposed corresponding to the tibialis anterior muscle of the lower leg L2 and applies electrical stimulation to the tibialis anterior muscle.

The detector 6 is fixed to the side surface of the right thigh L1 of the user U, and detects a motion state of the thigh L1. The detector 6 is, for example, an inertial measurement unit (IMU). The detector 6 of the present embodiment is a 9-axis IMU including a 3-axis acceleration sensor, a 3-axis gyro sensor, and a 3-axis compass. The detector 6 is connected to the controller 2 and gives the controller 2 an output including a detection result of each sensor. The controller 2 obtains an angular velocity of the thigh L1 as the motion state of the thigh L1. That is, the detector 6 functions as a sensor for detecting the angular velocity of the thigh L1.

The wearing tool 8 is a member for the user U wearing the electrodes 4 and the detector 6. The wearing tool 8 includes a pair of body portions 8a on the right and left. The body portions 8a are worn around the right and left legs L. Each of the body portions 8a is worn to cover the surfaces of the thigh L1 and the lower leg L2 of the leg L. That is, the body portion 8a is formed in a cylindrical shape like a supporter covering substantially the entire surface of the leg L. The body portion 8a is made of a stretchable material that is used for general supporters.

The first electrode 4_{BF}, the second electrode 4_{VM}, the third electrode 4_{So1}, and the fourth electrode 4_{TA} are provided on the inside surface of the body portion 8a. The body portion 8a holds each electrode 4 such that each electrode 4 is disposed at a predetermined position on the surface of the leg L when the body portion 8a is worn on the leg L. Further, the detector 6 is fixed to the outer surface of the body portion 8a worn on the right leg L. The right body portion 8a holds the detector 6 such that the detector 6 is disposed at a predetermined position of the leg L when the body portion 8a is worn on the leg L.

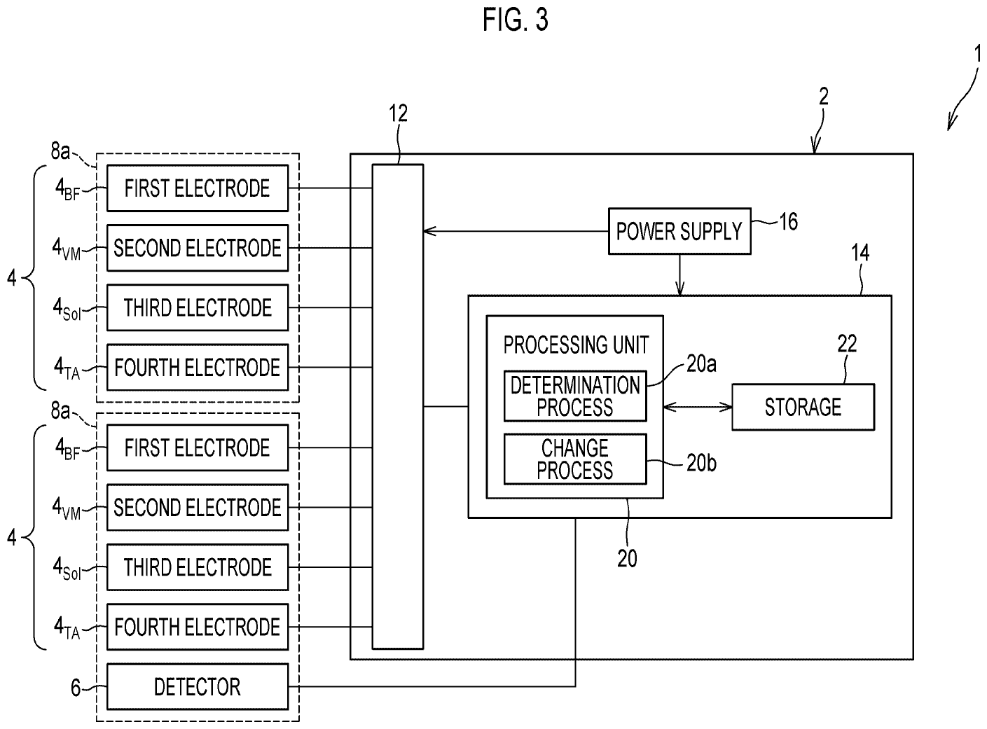

The controller 2 has a function of applying electrical stimulation to the muscles of the leg L by applying a voltage to each electrode 4. FIG. 3 is a block diagram showing a configuration of the controller 2. As shown in FIG. 3, the controller 2 includes a stimulation applier 12, a control unit 14, and a power supply 16. The power supply 16 is, for example, a battery, and supplies demanded power to the stimulation applier 12, the control unit 14, and the like.

The stimulation applier 12 has a function of applying, to the electrodes 4, a voltage for applying electrical stimulation to the muscles. Each electrode 4 and a power supply 16 are connected to the stimulation applier 12. The stimulation applier 12 includes a circuit that generates the voltage to be applied to the electrodes 4, a plurality of switches for connecting and disconnecting the electrodes 4, and the like. The circuit generates the voltage to be applied to the electrodes 4 based on electrical power supplied from the power supply 16. The stimulation applier 12 can turn on and off the switches to individually supply the voltage to the electrodes 4.

Electrical stimulation is applied to the muscles of the legs L by the stimulation applier 12 applying the voltage to the electrodes 4. The muscles of the legs L contract when electrical stimulation is applied. Thereby, the walking assist device 1 can move the muscles of the leg L of the user U to assist the user U in walking. The voltage applied to each electrode 4 is set to either a set voltage that is set in advance for each electrode 4 to provide an appropriate stimulation to the user U, or an intermediate voltage that is ½ of the set voltage. The stimulation applier 12 operates the switches based on the control of the control unit 14 to apply the voltage to the electrodes 4.

The control unit 14 is connected to the stimulation applier 12 and has a function of controlling the stimulation applier 12. The control unit 14 is built by a computer or the like. The detector 6 is also connected to the control unit 14. The output of the detector 6 is given to the control unit 14.

The control unit 14 includes a processing unit 20, such as a processor, and a storage 22, such as a memory or a hard disk. The storage 22 stores computer programs to be executed by the processing unit 20 and information needed. The processing unit 20 implements various processes of the processing unit 20 by executing a computer program stored in a computer-readable non-transitory recording medium, such as the storage 22.

The processing unit 20 has a function of executing a process of controlling the stimulation applier 12, a determination process 20a, and a change process 20b, by executing the computer program. The processing unit 20 controls the stimulation applier 12 such that the voltage is applied to one or more target electrodes. The one or more target electrodes are electrodes 4 selected from among the electrodes 4. The determination process 20a is a process of determining which of the motion phases included in the motion cycle of the leg L is the motion phase of the leg L during the walking motion based on the output of the detector 6. The change process 20b is a process of changing the combination of electrodes selected as target electrodes for each of the motion phases included in the motion cycle. The processes will be described later.

Regarding Motion Phase

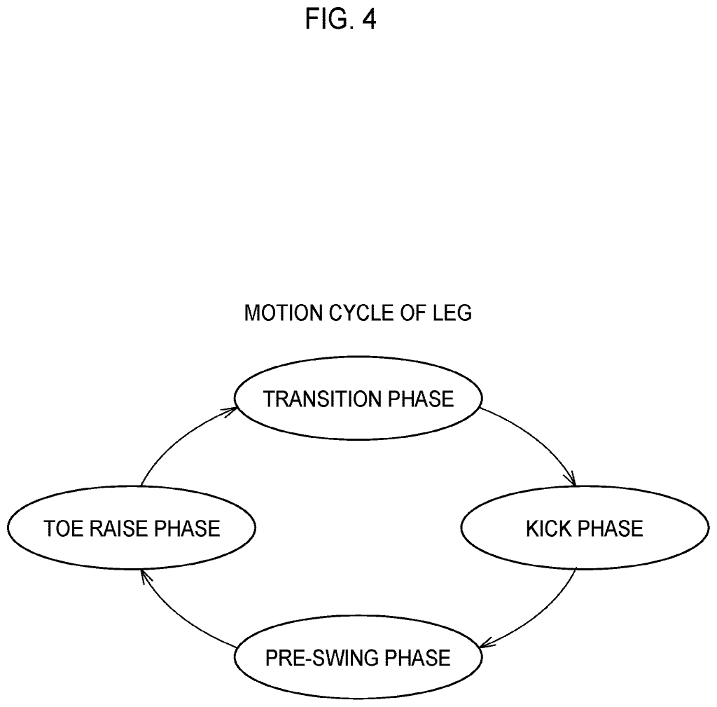

FIG. 4 is a diagram showing motion phases included in the motion cycle of the leg L. The control unit 14 of the present embodiment changes the electrodes 4 selected as the target electrode for each of the motion phases. The motion cycle of the leg L refers to one cycle of the leg L during the walking motion that moves periodically. The motion phase refers to a section obtained by dividing the motion cycle of the leg L into a plurality of sections according to the state of the leg L. As shown in FIG. 4, the motion cycle of the leg L includes a transition phase, a kick phase, a pre-swing phase, and a toe raise phase. When the user U is in a walking motion, each of the legs L repeats the four motion phases according to the order shown in FIG. 4.

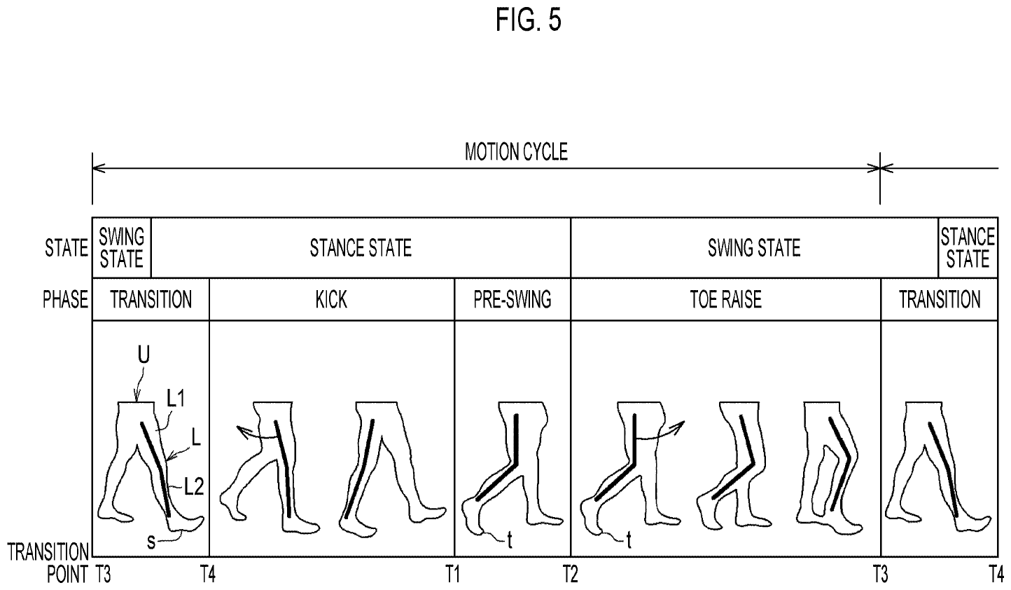

FIG. 5 is a diagram showing a state of the leg in four motion phases. In FIG. 5, the motion phases of the right leg L of the user U are shown. In FIG. 5, a drawn line attached along the right leg L of the user U shows an extending direction of the thigh L1 and the lower leg L2.

Among the four motion phases, the transition phase is a motion phase in which the leg L of the user U transitions from a swing state to a stance state. The swing state refers to a state in which the sole of the right leg L is off the ground. The stance state refers to a state in which at least part of the sole of the right leg L is in contact with the ground. The transition phase spans a period in which the leg L is in the swing state and a period in which the leg is in the stance state. In the transition phase, when the entire sole s of the leg L in the stance state touches the ground, the motion phase of the right leg L transitions from the transition phase to the kick phase. In the following description, the boundary between the transition phase and the kick phase will be referred to as transition point T4, as shown in FIG. 5.

The kick phase is a motion phase in which the leg L in the stance state is kicked backward. In the kick phase, the right leg L is in the stance state. Further, the kick phase includes a period in which the left leg L is in the swing state, and includes a period in which the user U stands with just the right leg L. In the kick phase, when the period of standing with just the right leg L ends (when the left leg L touches the ground), the motion phase of the right leg L transitions from the kick phase to the pre-swing phase. In the following description, the boundary between the kick phase and the pre-swing phase is referred to as transition point T1, as shown in FIG. 5.

The pre-swing phase is a motion phase in a state in which, immediately before the right leg L transitions from the stance state to the swing state, right and left legs are in contact with the ground. In the pre-swing phase, when a toe t of the right leg L leaves the ground and enters the swing state, the motion phase of the right leg L transitions from the pre-swing phase to the toe raise phase. In the following description, the boundary between the pre-swing phase and the toe raise phase is referred to as transition point T2, as shown in FIG. 5.

The toe raise phase is a motion phase in which the toe t of the leg L in the swing state is raised. In the toe raise phase, the user U swings the right leg L forward from the back in the state of raising the toe t. In the toe raise phase, when the lower leg L2 (tibia) of the leg L in the swing state becomes vertical, the motion phase of the right leg L transitions to the transition phase at that timing. In the following description, the boundary between the toe raise phase and the transition phase is referred to as transition point T3, as shown in FIG. 5.

As described above, the motion cycle of the right leg L includes four motion phases. The motion cycle of the left leg L, like the motion cycle of the right leg L, also includes four motion phases. In the motion cycle of the left leg L, transition point T1 in FIG. 5 is the boundary of the transition from the swing state to the stance state. Therefore, the motion cycle of the left leg L is shifted from the motion cycle of the right leg L by a predetermined timing. Therefore, when the timing of each motion phase of the right leg L can be grasped, the timing of each motion phase of the left leg L can also be grasped.

The walking assist device 1 of the present embodiment performs walking assist for the user U based on the four motion phases. Therefore, the processing unit 20 of the control unit 14 executes the determination process 20a for determining the motion phase of the leg L during the walking motion, and controls the stimulation applier 12 based on the determination result.

Regarding Determination Process

As described above, the determination process 20a (FIG. 3) is a process of determining the motion phase of the leg L during the walking motion based on the output of the detector 6. The processing unit 20 obtains an angular velocity ω of the right thigh L1, which is the motion state of the right thigh L1, based on the output from the detector 6. The processing unit 20 acquires the output from the detector 6 over time at predetermined sampling intervals, and obtains the angular velocity ω based on the acquired output. In the storage 22 of the processing unit 20, a relative position between the hip joint (rotation center of the thigh L1) of the user U and the detector 6 is stored in advance. The processing unit 20 obtains the angular velocity ω of the thigh L1 based on the relative position and the output of the detector 6. The processing unit 20 obtains the angular velocity ω as temporally continuous discrete values (time-series data). The processing unit 20 stores the obtained time-series data for the angular velocity ω in the storage 22.

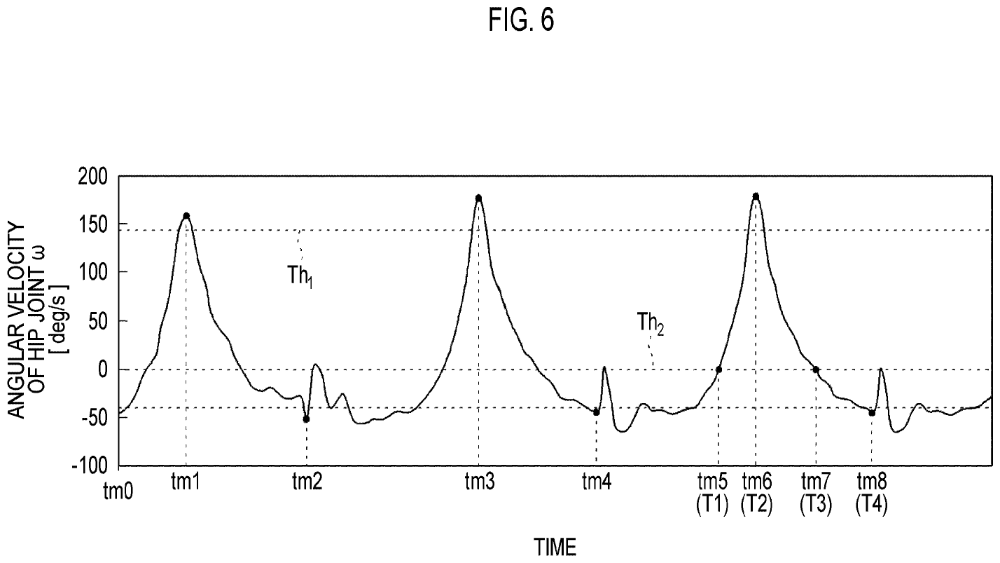

FIG. 6 is a graph showing an example of temporal changes in the angular velocity ω of the right thigh L1 obtained by the processing unit 20. In FIG. 6, the vertical axis is the angular velocity ω, and the horizontal axis is time. In FIG. 6, the angular velocity ω when the right thigh L1 rotates forward is indicated by a plus sign, and the angular velocity ω when the right thigh L1 rotates backward is indicated by a minus sign. That is, the minus sign attached to the angular velocity ω in FIG. 6 indicates the rotation direction, and does not indicate that the value of the angular velocity ω is a minus value. Therefore, in the following description, when the angular velocity ω is treated as a velocity, the angular velocity ω is treated as a positive value even if the minus sign is attached.

The processing unit 20 also obtains a differential value (angular acceleration) of the angular velocity ω over time. The processing unit 20 obtains a value obtained by subtracting a past angular velocity ωp by one sampling interval from the current (most recent) angular velocity ω, as a current (most recent) differential value. The processing unit 20 determines the motion phase of the leg L based on the angular velocity co obtained from the output of the detector 6 and the differential value.

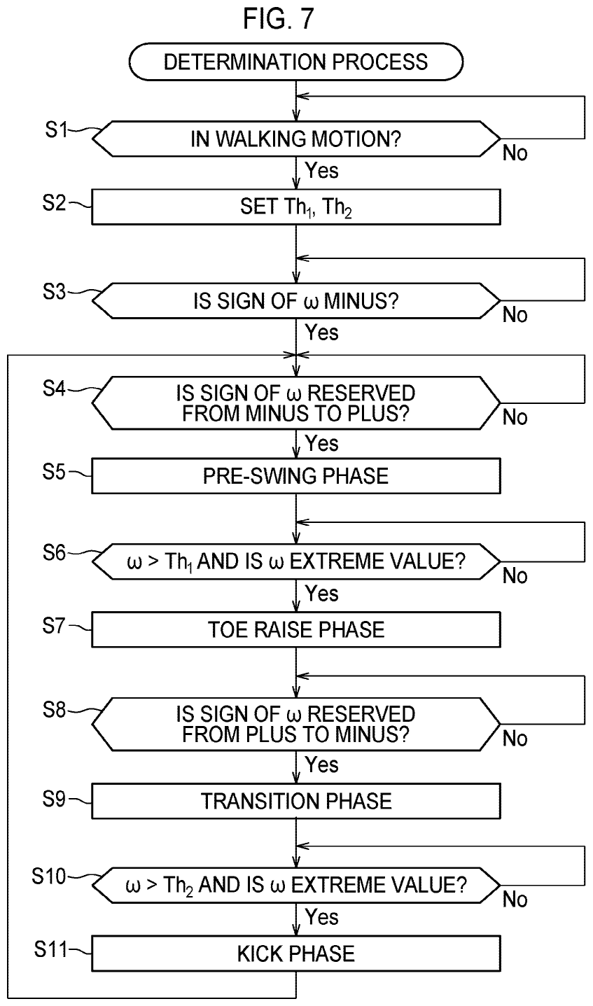

FIG. 7 is a flowchart showing an example of the determination process. In the determination process 20a, first, the processing unit 20 determines whether or not the user U is in a walking motion (step S1 in FIG. 7). The processing unit 20 determines whether or not the user U is in the walking motion based on the temporal change of the angular velocity ω.

In FIG. 6, in the graph showing the angular velocity Co, extreme values on the plus side appear at times tm1, tm3, and tm6. The extreme values are spaced at approximately constant intervals. The timing of the extreme values is the time to be determined as transition point T2, as will be described later. In this way, when a plurality of extreme values of the angular velocity ω appear at regular intervals in the angular velocity ω, a determination can be made that the thigh L1 is swinging, and that the user U is walking.

Therefore, the processing unit 20 determines that the user U is walking when two extreme values on the plus side appear in the time-series data for the angular velocity ω with predetermined intervals (step S1 in FIG. 7). The processing unit 20 repeats step S1 until the determination is made that the user U is walking.

For example, in FIG. 6, assuming that the user U starts walking at time tm0, then the processing unit 20 determines that the angular velocity ω at time tm1 and time tm3 is the extreme value on the plus side. The processing unit 20 determines that the most recent angular velocity ω when the value of the differential value of the angular velocity ω changes from positive to negative is the extreme value. Since the second extreme value on the plus side appears at time tm3, the processing unit 20 determines that the user U is in the walking motion at time tm3.

As shown in FIG. 7, when the determination is made that the user U is in the walking motion, the processing unit 20 proceeds to step S2 and sets a first threshold value Th1 and a second threshold value Th2 (step S2 in FIG. 7). In step S2, the processing unit 20 acquires two extreme values on the minus side in addition to the two extreme values on the plus side, sets the first threshold value Th1 based on the two extreme values on the plus side, and sets the second threshold value Th2 based on the two extreme values on the minus side. The first threshold value Th1 is a threshold value used to determine transition point T2 based on the angular velocity ω, as will be described later. The second threshold value Th2 is a threshold value used to determine transition point T4 based on the angular velocity ω, as will be described later.

For example, when the determination is made that the user U is in the walking motion at time tm3 in FIG. 6, the processing unit 20 sets the first threshold value Th1 based on the extreme value at time tm1 and the extreme value at time tm3 (step S2 in FIG. 7). For example, the processing unit 20 sets the first threshold value Th1 to a value that is smaller than the average value of the extreme value at time tm1 and the extreme value at time tm3 by a predetermined value. Further, the processing unit 20 may set the first threshold value Th1 to a value that is lower than the extreme value at time tm1 or the extreme value at time tm3, whichever is smaller, by a predetermined value.

The extreme value at time tm2 and the extreme value at time tm4 in FIG. 6 are used as the two extreme values on the minus side used to set the second threshold value Th2. The timing of the extreme values is the time to be determined as transition point T4, as will be described later. The extreme value on the minus side for setting the second threshold value Th2 is the maximum value of the angular velocity ω immediately before the angular velocity ω abruptly decreases to almost “0” (when the thigh L1 is almost stopped) after the angular velocity ω decelerates from the plus side and becomes “0” (the thigh L1 stops temporarily) and the angular velocity ω further enters the minus side (after the thigh L1 starts rotating backward).

After determining that the user U is in the walking motion at time tm3, the processing unit 20 acquires the second extreme value on the minus side when time tm4 is reached. Therefore, when time tm4 is reached, the processing unit 20 sets the second threshold value Th2 by using the extreme value at time tm2 and the extreme value at time tm4.

For example, the processing unit 20 sets a value smaller than the average value of the extreme value at time tm2 and the extreme value at time tm4 by a predetermined value (represented as an apparently large value in FIG. 6) to the second threshold value Th2. Further, the processing unit 20 may set a value lower by a predetermined value than the smaller of the extreme value of time tm2 and the extreme value of time tm4 (closer to “0”) to the second threshold value Th2.

When both threshold values Th1, Th2 are set, the processing unit 20 proceeds to step S3 in FIG. 7 and determines whether or not the sign of the angular velocity ω is minus (step S3 in FIG. 7). That is, the processing unit 20 determines whether or not the thigh L1 is rotating backward. The processing unit 20 repeats step S3 until the determination is made that the sign of the angular velocity ω is minus.

When the determination is made that the sign of the angular velocity ω is minus, then, the processing unit 20 determines whether or not the sign of the angular velocity ω is reversed from minus to plus (step S4 in FIG. 7). The processing unit 20 repeats step S4 until the determination is made that the sign of the angular velocity ω is reversed from minus to plus. When the determination is made in step S4 that the sign of the angular velocity ω is reversed from minus to plus, the processing unit 20 proceeds to step S5, and determines that the current point of time is transition point T1 (FIG. 5) and determines that the current motion phase of the leg L is the pre-swing phase (step S5 in FIG. 7).

In the kick phase, when the period of standing with just the right leg L ends, the motion phase of the right leg L transitions to the pre-swing phase. That is, transition point T1 is the timing at which the leg L ends rotating backward and switches to rotating forward. Therefore, the processing unit 20 can determine transition point T1 in step S4.

After the determination is made in step S5 that the motion phase is the pre-swing phase, the processing unit 20 proceeds to step S6 and determines whether or not the angular velocity ω is greater than the first threshold value Th1 and whether or not the angular velocity ω is an extreme value on the plus side (step S6 in FIG. 7). The processing unit 20 repeats step S6 until the determination is made that the angular velocity co is greater than the first threshold value Th1 and that the angular velocity ω is an extreme value on the plus side.

When the determination is made in step S6 that the angular velocity ω is greater than the first threshold value Th1 and that the angular velocity ω is an extreme value on the plus side, the processing unit 20 proceeds to step S7, and determines that the current point of time is transition point T2 (FIG. 5) and determines that the current motion phase is the toe raise phase (step S7 in FIG. 7).

In the pre-swing phase, when the leg L enters the swing state, the motion phase of the right leg L transitions to the toe raise phase. When the leg L enters the swing state, the leg L takes a motion like moving upward, and the angular velocity ω begins to decrease due to the load of the motion. Therefore, the processing unit 20 can determine transition point T2 in step S6.

After a determination is made that the motion phase is the toe raise phase in step S7, the processing unit 20 proceeds to step S8, and determines whether or not the sign of the angular velocity ω is reversed from plus to minus (step S8 in FIG. 7). The processing unit 20 repeats step S8 until the determination is made that the sign of the angular velocity ω is reversed from plus to minus.

When the determination is made in step S8 that the sign of the angular velocity ω is reversed from minus to plus, the processing unit 20 proceeds to step S9, and determines that the current point of time is transition point T3 (FIG. 5) and determines that the current motion phase of the leg L is the transition phase (step S9 in FIG. 7).

In the toe raise phase, when the lower leg L2 of the leg L in the swing state becomes vertical, the motion phase of the right leg L transitions to the transition phase. When the lower leg L2 of the leg L becomes vertical, the forward rotation of the leg L is almost ended, and then the leg L starts rotating backward. Therefore, the processing unit 20 can determine transition point T3 in step S8.

After the determination is made in step S9 that the motion phase is the transition phase, the processing unit 20 proceeds to step S10 and determines whether or not the angular velocity ω is greater than the second threshold value Th2 and whether the angular velocity ω is an extreme value on the minus side (step S10 in FIG. 7). The processing unit 20 repeats step S10 until the determination is made that the angular velocity ω is greater than the second threshold value Th2 and that the angular velocity ω is an extreme value on the minus side.

When the determination is made in step S10 that the angular velocity ω is greater than the second threshold value Th2 and that the angular velocity ω is an extreme value on the minus side, the processing unit 20 proceeds to step S11, and determines that the current point of time is transition point T4 (FIG. 5) and determines that the current motion phase is the kick phase (step S11 in FIG. 7).

In the transition phase, when the entire sole of the leg L in the stance state touches the ground, the motion phase of the right leg L transitions from the transition phase to the kick phase. When the entire sole of the leg L in the stance state touches the ground and the leg L is kicked backward, the angular velocity ω begins to decrease due to the load of the kick motion. Therefore, the processing unit 20 can determine transition point T4 in step S10.

After a determination is made in step S11 that the motion phase is the kick phase, the processing unit 20 returns to step S4 and repeats operations as described above.

Assuming that the processing unit 20 sets both threshold values Th1, Th2 at time tm4 in FIG. 6, the processing unit 20 then determines whether the sign of the angular velocity ω is minus (step S3 in FIG. 7). The sign of the angular velocity ω immediately after time tm4 is minus. Therefore, the processing unit 20 determines that the sign of the angular velocity ω is minus. Next, the processing unit 20 determines whether or not the sign of the angular velocity ω is reversed from minus to plus (step S4 in FIG. 7).

Then, when time tm5 is reached, the angular velocity ω passes through “0” from the minus side to the plus side as shown in FIG. 6. Therefore, the sign of the angular velocity ω is reversed from minus to plus at time tm5. Therefore, the processing unit 20 determines that time tm5 is transition point T1, and determines that the motion phase has transitioned to the pre-swing phase at time tm5 (steps S4, S5 in FIG. 7). In FIG. 6, when time tm6 is reached after time tm5, the angular velocity ω is greater than the first threshold value Th1 and becomes an extreme value on the plus side. Therefore, the processing unit 20 determines that time tm6 is transition point T2, and determines that the motion phase has transitioned from the pre-swing phase to the toe raise phase at time tm6 (steps S6, S7 in FIG. 7).

In FIG. 6, when time tm7 is reached after time tm6, the angular velocity ω passes through “0” from the plus side toward the minus side. Therefore, the sign of the angular velocity ω is reversed from plus to minus at time tm7. Therefore, the processing unit 20 determines that time tm7 is transition point T3, and determines that the motion phase has transitioned from the toe raise phase to the transition phase at time tm7 (steps S8, S9 in FIG. 7). In FIG. 6, when time tm8 is reached after time tm7, the angular velocity ω is greater than the second threshold value Th2 and becomes an extreme value on the minus side. Therefore, the processing unit 20 determines that time tm8 is transition point T4, and determines that the motion phase has transitioned from the toe raise phase to the transition phase at time tm8 (steps S10, S11 in FIG. 7).

When the user U is in the walking motion, the angular velocity ω repeats a waveform similar to a waveform from time tm4 to time tm8 in FIG. 6. Therefore, while the user U is in the walking motion, the processing unit 20 can continuously determine the motion phase of the right leg L based on the angular velocity ω. Further, the processing unit 20 also determines the motion phase of the left leg L by determining the motion phase of the right leg L.

As described above, in the present embodiment, the detector 6 that detects the motion state of the leg L, such as the angular velocity ω, is provided, and thus the motion phase of the leg L during the walking motion can be determined based on the motion state of the leg L.

Regarding Change Process

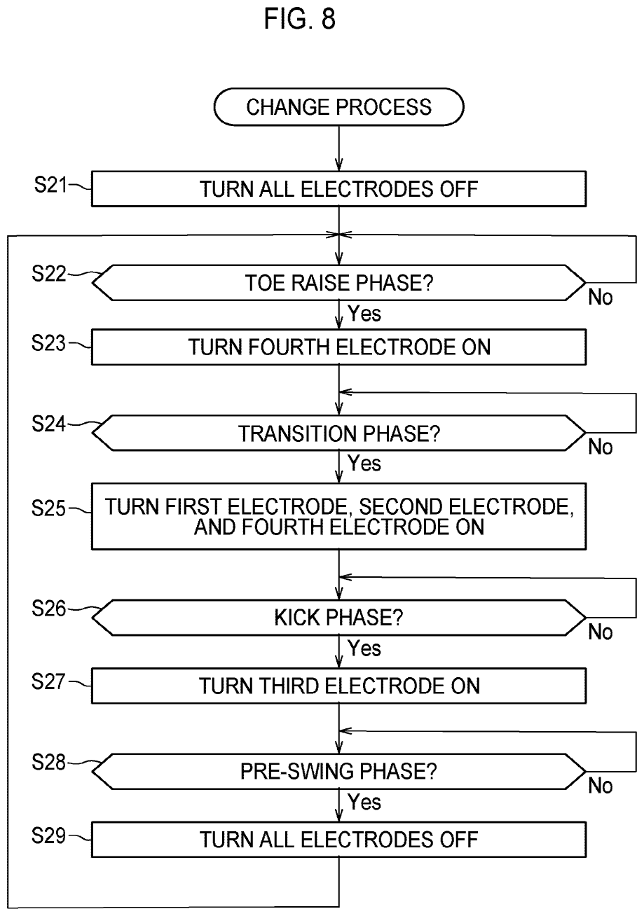

The processing unit 20 also executes the change process 20b (FIG. 3) in parallel with the determination process 20a (FIG. 3). As described above, the change process 20b is a process of changing the combination of the electrodes 4 selected as target electrodes for each of the four motion phases. A target electrode is an electrode 4 to which a voltage is applied from the stimulation applier 12. The processing unit 20 controls the stimulation applier 12 by the change process 20b such that the combination of the electrodes 4 selected as target electrodes is different for each of the four motion phases. FIG. 8 is a flowchart showing an example of the change process 20b. FIG. 8 shows the change process for the four electrodes 4 worn on one of the right and left legs L of the user U.

The processing unit 20 starts executing the determination process 20a and executing the change process 20b. First, the processing unit 20 brings the four electrodes 4 into a state in which no voltage is applied (step S21 in FIG. 8). In the following description, the state of the electrodes 4 to which no voltage is applied is called OFF, and the state of the electrodes 4 to which the voltage (set voltage) is applied is called ON. That is, the electrodes 4 in an ON state are selected as the target electrodes. The electrodes 4 in an OFF state are not selected as target electrodes.

After step S21, the processing unit 20 determines whether or not the determination result of the current motion phase of the leg L is the toe raise phase (step S22 in FIG. 8). The processing unit 20 repeats step S22 until the determination result of the current motion phase indicates the toe raise phase. When the determination result of the current motion phase indicates the toe raise phase, the processing unit 20 turns the fourth electrode 4_{TA} ON and keeps the other electrodes 4 OFF, among the four electrodes 4 (step S23 in FIG. 8). Therefore, in the toe raise phase, the processing unit 20 selects the fourth electrode 4_{TA} as the target electrode.

Next, the processing unit 20 determines whether or not the determination result of the current motion phase indicates the transition phase (step S24 in FIG. 8). The processing unit 20 repeats step S24 until the determination result of the current motion phase indicates the transition phase. When the determination result of the current motion phase indicates the transition phase, the processing unit 20 turns the first electrode 4_{BF}, the second electrode 4_{VM}, and the fourth electrode 4_{TA} ON, and keeps the third electrode 4_{So1} OFF, among the four electrodes 4 (step S25 in FIG. 8). Therefore, in the transition phase, the processing unit 20 selects the first electrode 4_{BF}, the second electrode 4_{VM}, and the fourth electrode 4_{TA} as target electrodes.

Next, the processing unit 20 determines whether or not the determination result of the current motion phase indicates the kick phase (step S26 in FIG. 8). The processing unit 20 repeats step S26 until the determination result of the current motion phase indicates the kick phase. When the determination result of the current motion phase indicates the kick phase, the processing unit 20 turns the third electrode 4ₛₒₗ ON, and turns the other electrodes 4 OFF, among the four electrodes 4 (step S27 in FIG. 8). Therefore, in the kick phase, the processing unit 20 selects the third electrode 4ₛₒₗ as the target electrode.

Next, the processing unit 20 determines whether or not the determination result of the current motion phase indicates the pre-swing phase (step S28 in FIG. 8). The processing unit 20 repeats step S28 until the determination result of the current motion phase indicates the pre-swing phase. When the determination result of the current motion phase indicates the pre-swing phase, the processing unit 20 turns all four electrodes 4 OFF (step S29 in FIG. 8). Therefore, in the pre-swing phase, the processing unit 20 does not set any of the electrodes 4 as the target electrode. Then, the processing unit 20 returns to step S22, and repeats each step thereafter.

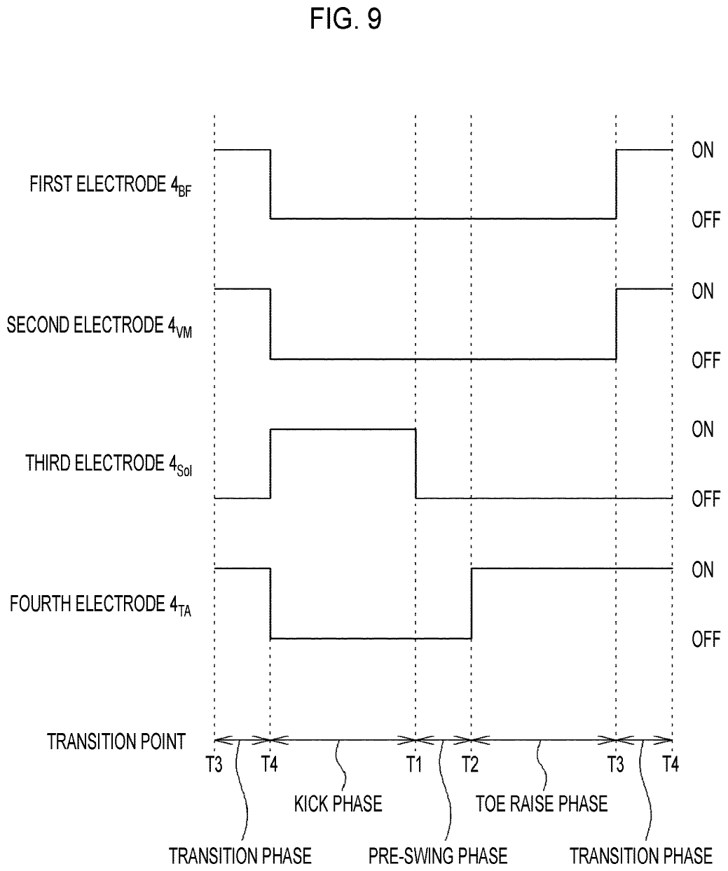

FIG. 9 is a time chart showing a state of each of the electrodes 4 controlled by the change process 20b described above. In FIG. 9, “OFF” indicates the potential when no voltage is applied to each electrode 4. “ON” indicates the potential when the set voltage is applied to each electrode 4. As shown in FIG. 9, the processing unit 20 controls the stimulation applier 12 such that the combination of the electrodes 4 selected as target electrodes is different for each of the four motion phases.

The processing unit 20 independently performs the change process for four electrodes 4 worn on the right leg L and the change process for four electrodes 4 worn on the left leg L. That is, the processing unit 20 performs the change process for the four electrodes 4 worn on the right leg L based on the determination result of the motion phase of the right leg L, and performs the change process for the four electrodes 4 worn on the left leg L based on the determination result of the motion phase of the left leg L.

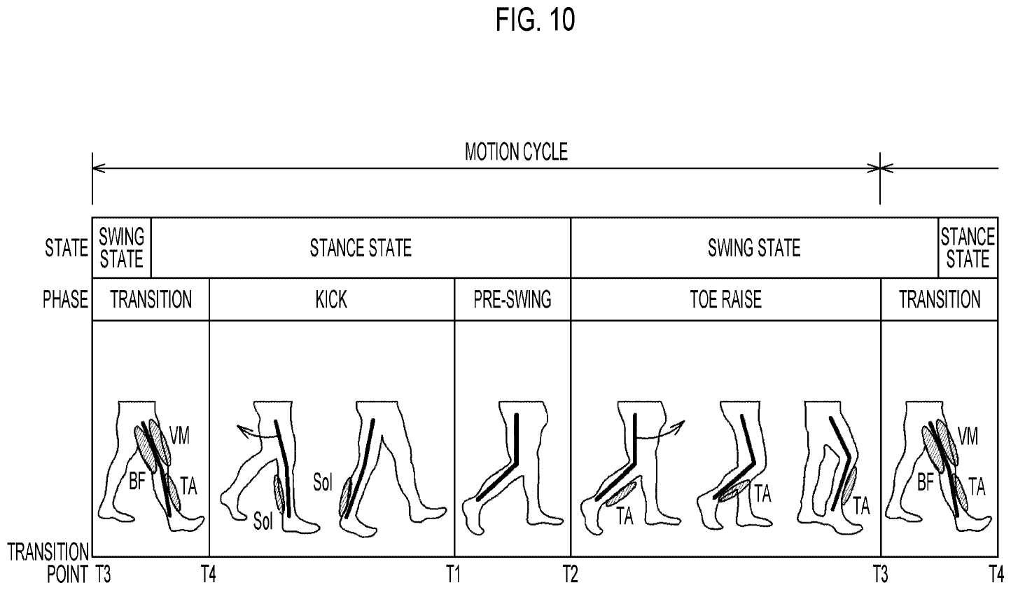

FIG. 10 is a diagram showing muscles to which electrical stimulation is applied when the electrodes 4 are controlled according to the time chart of FIG. 9, showing muscles to which electrical stimulation is applied for each motion phase. FIG. 10 shows muscles to which electrical stimulation is applied by the four electrodes 4 worn on the right leg L.

In the transition phase, the first electrode 4_{BF}, the second electrode 4_{VM} and the fourth electrode 4_{TA} are turned ON. Therefore, as shown in FIG. 10, electrical stimulation is applied to a biceps femoris muscle BF, a vastus medialis muscle VM, and a tibialis anterior muscle TA of the leg L. In the kick phase, the third electrode 4ₛₒₗ is turned ON. Therefore, as shown in FIG. 10, electrical stimulation is applied to the soleus muscle So1 of the leg L. In the pre-swing phase, all four electrodes 4 are turned OFF. Therefore, as shown in FIG. 10, electrical stimulation is not applied to the muscles of the legs L. In the toe raise phase, the fourth electrode 4_{TA} is turned ON. Therefore, as shown in FIG. 10, electrical stimulation is applied to the tibialis anterior muscle TA of the leg L. As shown in FIG. 10, electrical stimulation is also applied to the muscles of the left leg L.

As describe above, according to the present embodiment, the combination of the electrodes 4 selected as the target electrodes to which the voltage is applied is different for each of the four motion phases, and thus electrical stimulation can be selectively applied to a plurality of muscles of the leg L to be moved during the walking motion. As a result, walking assist can be performed appropriately.

In addition, in the present embodiment, the electrodes 4 are disposed corresponding to the biceps femoris muscle, the vastus medialis muscle, the soleus muscle, and the tibialis anterior muscle, and thus electrical stimulation can be applied to the appropriate muscles at appropriate timing during the walking motion.

Regarding Another Embodiment

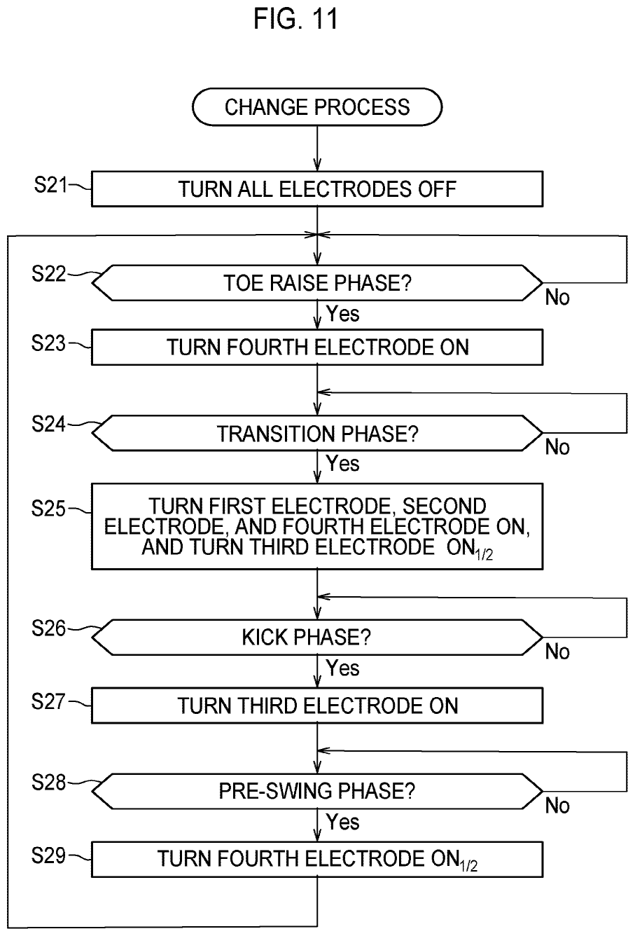

FIG. 11 is a flowchart showing an example of a change process according to another embodiment. The change process of the present embodiment differs from the above embodiment in that all the electrodes 4 are turned ON in step S25 and the fourth electrode is turned ON in step S29. Other points are the same as the above embodiment.

In FIG. 11, when the determination result of the current motion phase indicates the transition phase (step S24), the processing unit 20 of the present embodiment proceeds to step S25 and turns all four electrodes 4 ON. Therefore, in the transition phase, the processing unit 20 selects all four electrodes 4 as target electrodes. In this case, an intermediate voltage that is ½ of the set voltage is applied to the third electrode 4_{So1}. The set voltage is a voltage that is set in advance for each of the electrodes 4 such that the user U is appropriately stimulated, as described above. “ON_{1/2}” in FIG. 11 indicates the state of the electrode 4 to which the intermediate voltage is applied. Hereinafter, the state of the electrode 4 to which the intermediate voltage is applied is indicated as “ON_{1/2}”.

Next, the processing unit 20 determines whether or not the determination result of the current motion phase indicates the kick phase (step S26 in FIG. 11). The processing unit 20 repeats step S26 until the determination result of the current motion phase indicates the kick phase. When the determination result of the current motion phase indicates the kick phase, the processing unit 20 turns the third electrode 4ₛₒₗ ON, and turns the other electrodes 4 OFF, among the four electrodes 4 (step S27 in FIG. 11). Therefore, in the kick phase, the processing unit 20 selects the third electrode 4ₛₒₗ as the target electrode. In this case, the set voltage is applied to the third electrode 4_{So1}.

Next, the processing unit 20 determines whether or not the determination result of the current motion phase indicates the pre-swing phase (step S28 in FIG. 11). The processing unit 20 repeats step S28 until the determination result of the current motion phase indicates the pre-swing phase. When the determination result of the current motion phase indicates the pre-swing phase, the processing unit 20 turns the fourth electrode 4_{TA} ON_{1/2}, and turns the other electrodes 4 OFF, among the four electrodes 4 (step S29 in FIG. 8). Therefore, in the pre-swing phase, the processing unit 20 selects the fourth electrode 4_{TA} as the target electrode. In this case, the intermediate voltage is applied to the fourth electrode 4_{TA}. Then, the processing unit 20 returns to step S22, and repeats each step thereafter.

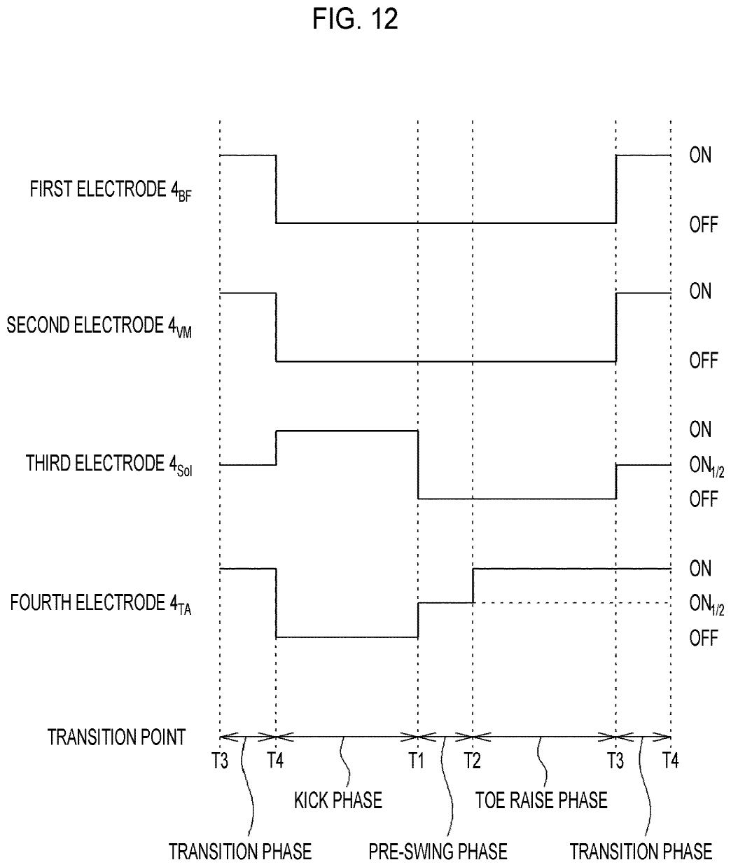

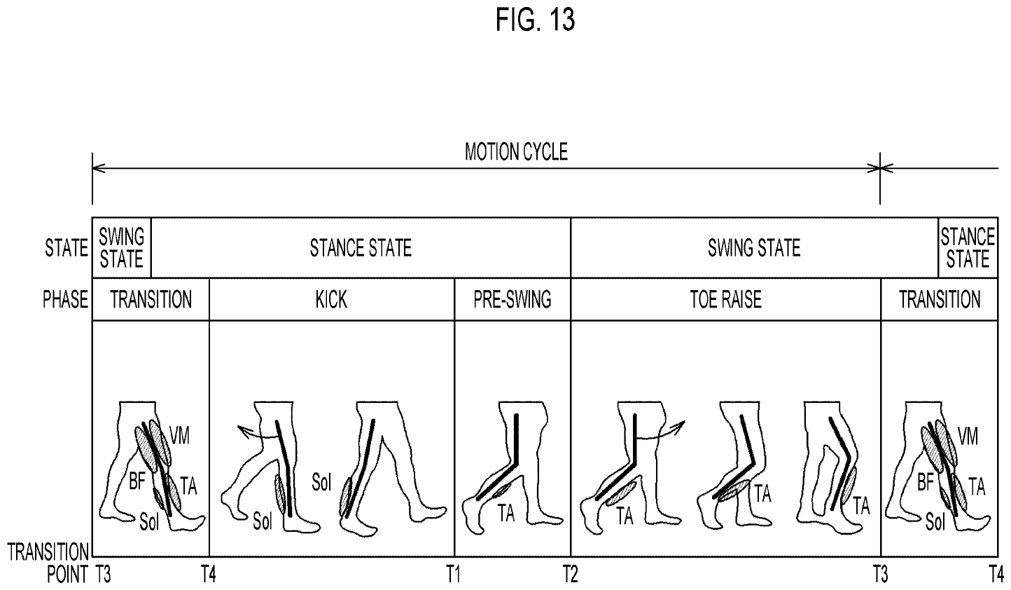

FIG. 12 is a time chart showing states of each of the electrodes 4 controlled by the change process according to the other embodiment. In FIG. 12, “OFF” indicates the potential when no voltage is applied to each electrode 4. “ON” indicates the potential when the set voltage is applied to each electrode 4. “ON_{1/2}” indicates the potential when the intermediate voltage is applied to each electrode 4. FIG. 13 is a diagram showing muscles to which electrical stimulation is applied when the electrodes 4 are controlled according to the time chart of FIG. 12. In the present embodiment, the processing unit 20 also controls the stimulation applier 12 such that the combination of the electrodes 4 selected as target electrodes is different for each of the four motion phases.

In the transition phase, the first electrode 4_{BF}, the second electrode 4_{VM}, and the fourth electrode 4_{TA} are turned ON, and the third electrode 4ₛₒₗ is turned ON_{1/2}. Therefore, as shown in FIG. 13, electrical stimulation is applied to the biceps femoris muscle BF, the vastus medialis muscle VM, the soleus muscle So1, and tibialis anterior muscle TA of the leg L. In the kick phase, the third electrode 4_{So1} is turned ON. Therefore, as shown in FIG. 13, electrical stimulation is applied to the soleus muscle So1 of the leg L.

The voltage applied to the third electrode 4ₛₒₗ is changed from the intermediate voltage to the set voltage when transition from the transition phase to the kick phase is made, and increases stepwise. In this way, the electrical stimulation to the soleus muscle So1 increases stepwise when the transition from the transition phase to the kick phase is made, and thus the muscles can be smoothly moved.

In the pre-swing phase, the fourth electrode 4_{TA} is turned ON_{1/2}. Therefore, as shown in FIG. 13, electrical stimulation is applied to the tibialis anterior muscle TA of the leg L. In the toe raise phase, the fourth electrode 4_{TA} is also turned ON. Therefore, as shown in FIG. 13, electrical stimulation is continuously applied to the tibialis anterior muscle TA of the leg L. The voltage applied to the fourth electrode 4_{TA} is changed from the intermediate voltage to the set voltage when the pre-swing phase transitions to the toe raise phase, and increases stepwise. In this way, the electrical stimulation to the tibialis anterior muscle TA is increased stepwise when the pre-swing phase transitions to the toe raise phase, and the muscle can be smoothly moved.

In the present embodiment, as aspect in which either the set voltage or the intermediate voltage is applied to the third electrode 4_{So1} and the fourth electrode 4_{TA}; however, as long as the intermediate voltage is lower than the set voltage, the intermediate voltage need not be ½ of the set voltage, and may be lower than ½ or higher than ½.

Others

The embodiments disclosed herein are illustrative in all respects and are not restrictive. For example, in the embodiments, the case has been illustrated in which the biceps femoris muscle, the vastus medialis muscle, the soleus muscle, the tibialis anterior muscle are targeted as the muscles used for the walking motion, and the electrodes 4 are disposed for applying electrical stimulation to the muscles; however, in addition to the muscles, other muscles used for the walking motion may also be targeted, and electrodes may be disposed thereon. Furthermore, some of the biceps femoris muscle, the vastus medialis muscle, the soleus muscle, and the tibialis anterior muscle, and muscles other than the muscles may also be targeted and electrodes may be disposed thereon.

Further, in the embodiments, the motion cycle of the leg L is divided into four motion phases; however, for motion phases, fewer division sections may be used, or five or more division sections may be used.

Further, in the embodiments, the case has been illustrated in which the detector 6 is provided on the right thigh L1; however, the detector may be provided on the left thigh L1 as well. In this case, the motion phases of the right and left legs L can be determined independently, and the motion phases can be determined more precisely. In addition, when the detector 6 is provided just on the right thigh L1 as in the embodiment, the motion phases of the right and left legs L can be determined by the output of the detector 6, and the configuration of the walking assist device 1 can be simplified and the cost can be reduced.

The scope of the disclosure is not limited to the above-described embodiments, and includes all modifications within the scope of equivalents to the configurations described in the claims.

What is claimed is:

1. A walking assist device comprising:
a plurality of electrodes disposed on a surface of a leg of a user and corresponding to a plurality of muscles used for a walking motion;
a stimulation applier configured to apply, to the electrodes, a voltage for applying electrical stimulation to the muscles; and
a control unit configured to control the stimulation applier to apply the voltage to one or more target electrodes selected from among the electrodes,
wherein the control unit is configured to control the stimulation applier such that a combination of electrodes selected as the one or more target electrodes is different for each of a plurality of motion phases included in a motion cycle of the leg during the walking motion.

2. The walking assist device according to claim 1, further comprising a detector configured to detect a motion state of the leg,
wherein the control unit is configured to perform a determination process for determining, based on an output of the detector, which of the motion phases is a current motion phase of the leg during the walking motion.

3. The walking assist device according to claim 2, wherein the motion state of the leg includes an angular velocity of a thigh of the leg.

4. The walking assist device according to claim 1, wherein the muscles include a biceps femoris muscle, a vastus medialis muscle, a soleus muscle, and a tibialis anterior muscle.

5. The walking assist device according to claim 1, wherein:
the motion phases include four motion phases of a transition phase, a kick phase, a pre-swing phase, and a toe raise phase;
the transition phase is a motion phase in which the leg transitions from a swing state to a stance state;
the kick phase is a motion phase in which the leg in the stance state kicks backward;
the pre-swing phase is a motion phase in a state in which, immediately before the leg transitions from the stance state to the swing state, right and left legs are in contact with a ground; and
the toe raise phase is a motion phase in which a toe of the leg in the swing state is raised.

6. The walking assist device according to claim 1, wherein:
the muscles include a biceps femoris muscle, a vastus medialis muscle, a soleus muscle, and a tibialis anterior muscle, and the motion phases include four motion phases of a transition phase, a kick phase, a pre-swing phase, and a toe raise phase;
the transition phase is a motion phase in which the leg transitions from a swing state to a stance state;
the kick phase is a motion phase in which the leg in the stance state kicks backward;
the pre-swing phase is a motion phase in a state in which, immediately before the leg transitions from the stance state to the swing state, right and left legs are in contact with a ground;
the toe raise phase is a motion phase in which a toe of the leg in the swing state is raised;
in the transition phase, electrodes disposed corresponding to the biceps femoris muscle, the vastus medialis muscle, and the tibialis anterior muscle among the electrodes are the target electrodes;
in the kick phase, an electrode disposed corresponding to the soleus muscle among the electrodes is the target electrode;
in the pre-swing phase, none of the electrodes is the target electrode; and
in the toe raise phase, an electrode disposed corresponding to the tibialis anterior muscle among the electrodes is the target electrode.

7. The walking assist device according to claim 1, wherein:
the muscles include a biceps femoris muscle, a vastus medialis muscle, a soleus muscle, and a tibialis anterior muscle;
the motion phases include four motion phases of a transition phase, a kick phase, a pre-swing phase, and a toe raise phase;
the transition phase is a motion phase in which the leg transitions from a swing state to a stance state;
the kick phase is a motion phase in which the leg in the stance state kicks backward;
the pre-swing phase is a motion phase in a state in which, immediately before the leg transitions from the stance state to the swing state, right and left legs are in contact with a ground;
the toe raise phase is a motion phase in which a toe of the leg in the swing state is raised;
in the transition phase, electrodes disposed corresponding to the biceps femoris muscle, the vastus medialis muscle, the soleus muscle, and the tibialis anterior muscle among the electrodes are the target electrodes;
in the kick phase, an electrode disposed corresponding to the soleus muscle among the electrodes is the target electrode;
in the pre-swing phase, an electrode disposed corresponding to the tibialis anterior muscle is the target electrode;
in the toe raise phase, the electrode disposed corresponding to the tibialis anterior muscle among the electrodes is the target electrode;
the voltage applied to the electrode disposed corresponding to the soleus muscle in the transition phase is lower than the voltage applied to the electrode disposed corresponding to the soleus muscle in the kick phase; and
the voltage applied to the electrode disposed corresponding to the tibialis anterior muscle in the pre-swing phase is lower than the voltage applied to the electrode disposed corresponding to the tibialis anterior muscle in the toe raise phase.
